(19) United States
(12) Patent Application Publication
(10) Pub. No.: US 2024/0042086 A1
(43) Pub. Date: Feb. 8, 2024

(54) Scented Air Filters

(71) Applicants: Danielle Melton, Spring, TX (US); David E. Melton, Spring, TX (US)

(72) Inventors: Danielle Melton, Spring, TX (US); David E. Melton, Spring, TX (US)

(21) Appl. No.: 18/202,235

(22) Filed: May 25, 2023

Related U.S. Application Data
(60) Provisional application No. 63/394,989, filed on Aug. 4, 2022.

(51) Int. Cl. A61L 9/12

(52) U.S. Cl. CPC A61L 9/12; A61L 2209/14

(57) ABSTRACT

Scented air filters for homes and vehicles are provided. The device includes a frame and a plurality of filter layers. The plurality of filter layers is connected to the frame. The plurality of filter layers is configured to filter the air that passes through each layer. The frame is designed to resemble standard home or car cabin air filters. The plurality of filter layers further includes a scented material disposed of thereon. The scented material is designed to enhance the scent of the air that is pushed through the air filter.

### BACKGROUND

Air purification is important for maintaining a healthy living or working environment. Most air includes molecules that can carry debris or even illnesses. When an individual breathes in unpurified air, they may be susceptible to the illnesses molecules and even have difficulty breathing. This is especially true in closed-off environments in a home or an automobile cabin. To limit the debris and illnesses that are contained within the unpurified air, individuals will have the air pass through at least one filter before it reaches the closed-off environment. In this way, the individual will assure themselves that all the air that they are breathing in will have already been properly filtered and the contaminated molecules have been mostly eliminated from the air. Additionally, air, both purified and unpurified, may contain an undesirable odor. Such an odor can be difficult to remove for the air molecules, even if the air passes through a filtration process. Many of the standard air purification devices that are available from homes and automobiles still allow the undeniable odor to enter those closed-off environments. Typically, the individual will have to utilize an additional device, such as an aerosol bottle, that can remove an undesirable odor from the air and replace it with a more desirable odor therein. In order to address these concerns, the present invention will provide users with an air filtration device with a fragrance that gets absorbed by the air passing through the air filtration device to leave the environment with a more desirable odor.

### DESCRIPTION OF DRAWINGS

FIG. 1 shows a top view of an embodiment of the scented air filter.

FIG. 2 shows a bottom view of an embodiment of the scented air filter.

### DESCRIPTION OF THE INVENTION

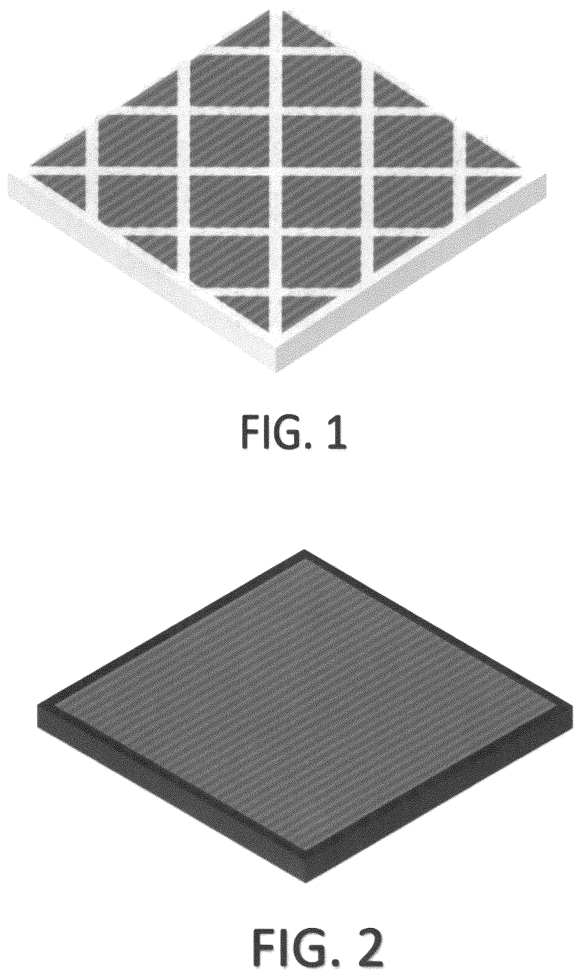

Referring now to FIGS. 1 and 2, there are shown a top view and a bottom view of alternative embodiments of a scented air filter, respectively. The scented air filter comprises a frame and a plurality of filter layers. The frame is designed to fit into standard home or car cabin air filtration devices. The frame has a plurality of sides and an interconnecting support.

The interconnecting support is disposed from the bottom face of the plurality of sides of the frame. The plurality of filter layers is connected to each side of the plurality of sides of the frame. The plurality of filter layers covers an internal opening of the frame. The interconnecting support allows the plurality of filter layers to maintain their integrity while the air passed through them. In one embodiment, the interconnecting support includes a crossing configuration. The plurality of filter layers is configured to filter unpurified air therethrough. A filtration system will transport to the scented air filter. The air will then enter the device through the plurality of filter layers. With each filter layer of the plurality of filter layers that the unpurified air passes through, more contaminants therein will be removed. When the air exits all the filter layers, the air will be purified and exit the filtration system into a desired environment, Additionally, the plurality of filter layers includes a scented material. The scent material is disposed of thereon to become an absorber in the air molecules. The scented material is designed to enhance the scent of the air that passes through the plurality of filter layers. This will leave the purified air that passed through the scented air filter to contain a desired fragrance to provide an aroma in the desired environment.

1. The unique features of Scented Air Condition Filters will provide the following benefits for consumers worldwide:
No other product, process, or service currently available provides a two-and-one product like Scented Air Condition Filters.
The AC/heater filter provides temperature control, traps dust, and particles, and adds freshness to the air at the same time.
Allows for the removal of air fresheners, plu-ins, candles, and other air freshening agents while also providing a range of scents to provide pleasant aromas into the environment.
Users will save money by being able to infiltrate a fresh scent into the air while also filtering the air without having to purchase extraneous and often detrimental devices to infiltrate scent into the air.
Scented Air Condition Filters stand alone—there is nothing like it on the market today.
